(19) United States
(12) Patent Application Publication
(10) Pub. No.: US 2026/0199153 A1
(43) Pub. Date: Jul. 16, 2026

(54) PRODUCT DISPENSING SYSTEM

(71) Applicant: Aunt Flow Corp., Columbus, OH (US)

(72) Inventors: Claire Coder, Perrysburg, OH (US); Gordon Thomas, Mason, OH (US); Mark Dalhart, Lebanon, OH (US)

(21) Appl. No.: 19/134,137

(22) PCT Filed: Nov. 30, 2022
(86) PCT No.: PCT/US2022/051447

§ 371 Date: May 29, 2025

(51) Int. Cl. A61F 15/00; B65D 83/02

(52) U.S. Cl. CPC A61F 15/003; B65D 83/02

(57) ABSTRACT

A product dispensing system. The system includes an outer housing that houses at least one dispensing unit configured to retain a quantity of product and dispense it when needed. A dispensing unit includes a chute attached to a chassis that is configured to hold a quantity of product, as well as a push-button activated gear assembly that is configured to move a moving element within the chassis to dispense a product. The dispensed product can be ejected out of the outer housing or retained within until it is retrieved.

### CROSS-REFERENCE TO RELATED APPLICATIONS

This application is a 371 filing of International Patent Application No. PCT/US2022/051447, entitled “PRODUCT DISPENSING SYSTEM,” filed Nov. 30, 2022, which is incorporated herein by reference in its entirety for all purposes.

### FIELD

The application relates to systems, devices, and methods for dispensing products, and in particular to systems, devices, and methods for dispensing personal care products (PCP) and personal protective equipment (PPE), such as menstrual products, sanitizing wipes, face masks, and gloves.

### BACKGROUND

Personal care products may be used for personal hygiene (e.g., to care for bodily health and wellbeing through cleanliness). For example, menstrual products can be used for personal hygiene during menstruation or other bodily functions. Common personal care products include menstrual products such as tampons and pads. Often, the need for personal care products may come unexpectedly. For example, menstrual flow may start unexpectedly. A lack of readily available personal care products can negatively affect personal health and self-esteem, may cause soiling of clothing, and may disrupt daily activities. A person that does not have readily available personal care products when there is a need may have to leave school or work or abstain from sports or other activities. Conventionally, to obtain personal care products, an individual has to travel to a store or to their residence.

Conventional personal care product dispensing systems use products that are individually packaged with robust packaging to withstand forces during transportation, loading of the dispensing system, dispensing of the product, and the like. For example, each individual product, such as a tampon or pad, may be packaged in a box. Pads are conventionally put in boxes that are around 25 mm thick. The robust individual packaging of each product requires extra room within the dispensing system and increases costs of each product and of the dispensing system. This often results in the need for a larger dispensing unit that can hold a sufficient number of products containing the robust packaging. When used, the individual packaging around each personal care product, including any robust packaging, is typically discarded and must enter the waste stream.

The use of personal protective equipment (PPE) is a way to lower an individuals risk of illness caused by viruses and other pathogens. The risk individuals face when they do not have proper PPE is especially high during flu season, pandemics such as the COVID-19 pandemic, or any other communicable disease outbreak. Various types of PPE, including sanitizing wipes, face masks, and gloves can offer individuals increased protection against germs and illness, particularly when they are in proximity to other persons, such as at work, school, or shopping. However, if an individual does not adequately plan ahead, they may find themselves without PPE and at an increased health risk, particularly when engaging in an activity that puts them in close proximity to others. It is often inconvenient or impossible for an individual to get to the proper type of store to purchase PPE while they are at work, school, traveling, shopping or engaging in other activities.

Conventional product dispensing systems may be difficult to load, difficult to timely restock, and may result in waste of products and packaging. For example, placing individual items into a conventional dispensing system is often time consuming and may cause damage to the individual items. The need to touch each individual item during restocking in order to place it into the dispenser may also cause health and cleanliness concerns.

Determining when to restock a conventional dispensing system may also be difficult and time consuming. Conventionally, dispensing systems require that one open the dispenser in order to view how much inventory is left. Accordingly, the depletion or absence of stock may often go unnoticed. Stock can be depleted quickly if users are allowed to dispense multiple products from the dispensing system within a short time period. Accordingly, it is very common for conventional dispensers to remain empty for long periods of time because they do not hold a sufficient amount of product, they are time consuming to refill, or it is otherwise not apparent when the dispenser is empty.

Conventionally, dispensing systems may become damaged from improper usage, such as the forcing of buttons and levers. Conventional dispensing systems are built robustly (e.g., made of metal and glass) to attempt to withstand improper usage. The increased cost to build and repair a conventional robust dispensing system is often transferred to the cost of the products. Even if a conventional dispensing system is available, users may not use the conventional dispensing system because of the overpriced products.

Lastly, conventional systems may take up a large amount of space in a bathroom or other area, creating an obstacle for people to walk around or a risk that someone will accidentally make contact with the system which may result in personal injury or damage to the system.

Accordingly, those skilled in the art continue with research and development efforts in the field of product dispensing systems.

### SUMMARY OF THE INVENTION

Disclosed are dispensing systems configured to retain a quantity of product and dispense single units of product. In one embodiment, a dispensing system comprises an outer housing having a front cover and a rear case, wherein the rear case defines and interior volume and the front cover defines a slot extending into the interior volume. A tray is located within the interior volume, proximate to the slot. Within the interior volume is housed a dispensing unit having a chassis. The chassis defines a top-side opening, an open front-side, and an interior space. The open front-side of the chassis may face the tray, and a product may be loaded into the chassis by being inserted into the top-side opening. A moving element is mounted to the chassis within the interior space and below the top-side opening, the moving element being moveable between at least a first position and a second position. Movement of the moving element from its first position to its second position causes the moving element to move a loaded product towards the tray through the open front side. A gear assembly is configured to move the moving element between its first and second positions. The gear assembly is comprised of a drive spur gear fixedly connected to the moving element, a rack gear slidably attached to the chassis that is moveable along a linear path, and at least one additional spur gear meshed with the drive spur gear and the rack gear. The at least one additional spur gear is configured to translate linear motion by the rack gear into rotational motion of the drive spur gear.

The movement element may be a rocker component that comprises a linear shaft rotatably mounted to the chassis below the top-side opening and a linear section extending transverse to the linear shaft. In a first position the linear section is oriented upwards towards the rear of the chassis, and in a second position, the linear section is oriented upwards towards the open front side. Movement of the rocker component from its first position to its second position causes the linear section to push a loaded product towards the tray through the open front side. The drive spur gear is fixedly connected to the linear shaft and is configured to rotate the linear shaft when it is rotated. In an exemplary embodiment, the linear section of the rocker component defines a distal end, and the rocker component comprises a rolling element connected to the distal end. In an exemplary embodiment, the rocker component includes a forward protrusion extending from the linear section towards the front side of the chassis. The forward protrusion is configured to support a loaded product from beneath. In an exemplary embodiment, the rocker component comprises a torsion spring fitted over the linear shaft that is configured to urge the rocker component towards its first position. In an exemplary embodiment, the chassis comprises a rail extending into the interior space near the top-side opening. The rail is configured to support a loaded product from beneath.

In an exemplary embodiment of the dispensing system, the moving element is a roller component that comprises a cylindrical portion that defines opposing ends and a curved exterior surface. The roller component further comprises opposing axial portions extending from the ends of the cylindrical portion that are rotatably mounted to the chassis, and a groove defined in the exterior surface of the cylindrical portion. In the first position, the groove is oriented upwards towards the top-side opening of the chassis and is thereby able to receive a loaded product. In the second position, the groove is oriented at least slightly downwards towards the front open side of the chassis. Movement of the roller component from its first position to its second position causes the cylindrical portion to delivery a product from the top-side opening to the open front side, causing the product to fall out of the groove towards the tray. The drive spur gear is fixedly connected to an axial portion of the roller component and is configured to rotate the axial portion when it is rotated.

In an exemplary embodiment, the roller component comprises a plurality of ribs within the groove that limit the number of products capable of fitting within the groove.

In an exemplary embodiment, the chassis comprises a ramp located along its open front side that defines a rear-to-front downward slope and is configured to direct a product falling out of the groove towards the tray.

In an exemplary embodiment, the slot defined in the front cover defines a lower edge, and the tray is flush with the lower edge of the slot. In another exemplary embodiment, the tray is located below the lower edge.

In an exemplary embodiment, the outer housing defines an opening into the interior volume, and the dispensing system comprises a button that is attached to the rack gear and fitted to the opening defined by the outer housing. In an exemplary embodiment, the button is spring-backed and configured to move the rack gear towards one direction along its linear path.

In an exemplary embodiment, the dispensing unit further comprises an elongated chute located above the chassis that is configured to hold a quantity of product and gravity-feed the product into the top-side opening. In an exemplary embodiment, the chute defines a front-side cutout extending along its length.

Other examples of the disclosed dispensing system, and methods of using and making the same, will become apparent from the following detailed description, the accompanying drawings, and the appended claims.

### BRIEF DESCRIPTION OF THE DRAWINGS

FIG. 1 is a front perspective view of an exemplary embodiment of a product dispensing system;

FIG. 2 is a rear perspective view of the system of FIG. 1;

FIG. 3 is a front perspective view of the front cover and paneling of the system of FIG. 1 shown in isolation;

FIG. 4 is a rear perspective view of the front cover and paneling of FIG. 3;

FIG. 5 is a front perspective view of the rear case of the system of FIG. 1 shown in isolation;

FIG. 6 is a front elevation view of the system of FIG. 1 with the front cover and paneling removed;

FIG. 7 is a front perspective view of the first dispenser unit of the system of FIG. 1 shown in isolation;

FIG. 8 is a rear perspective view of the first dispenser unit of FIG. 7;

FIG. 9 is another rear perspective view of the first dispenser unit of FIG. 7;

FIG. 10 is another front perspective view of the first dispenser unit of FIG. 7;

FIG. 11 is a front perspective view of the chassis of the first dispenser unit of FIG. 7 shown in isolation;

FIG. 12 is a rear perspective view of the chassis of FIG. 11;

FIG. 13 is a top plan view of the chassis of FIG. 11;

FIG. 14 is a front perspective view of the rocker component of the first dispenser unit of FIG. 7;

FIG. 15 is a left-side cross-sectional view of the chassis and rocker component of the first dispenser unit of FIG. 7;

FIG. 16 is a right-side cross-sectional view of the chassis and rocker component of the first dispenser unit of FIG. 7;

FIG. 17 is a left-side elevation view of the chassis and gear assembly of the first dispenser unit of FIG. 7;

FIG. 18 is a front perspective view of the second dispenser unit of the system of FIG. 1 shown in isolation;

FIG. 19 is a rear perspective view of the second dispenser unit of FIG. 18;

FIG. 20 is another rear perspective view of the second dispenser unit of FIG. 18;

FIG. 21 is another front perspective view of the second dispenser unit of FIG. 18;

FIG. 22 is a front perspective view of the chassis of the second dispenser unit of FIG. 18;

FIG. 23 is a right-side perspective view of the chassis of FIG. 22;

FIG. 24 is a front elevation view of the roller component of the second dispenser unit of FIG. 18;

FIG. 25 is a right-side perspective view of the roller component of FIG. 24;

FIG. 26 is a right-side cross-sectional view of the chassis and roller component of the second dispenser unit of FIG. 18;

FIG. 27 is a left-side cross-sectional view of the chassis and roller component of the second dispenser unit of FIG. 18;

FIG. 28 is a right-side elevation view of the chassis and gear assembly of the second dispenser unit of FIG. 18;

FIG. 29 is a front elevation view of a menstrual pad product; and

FIG. 30 is front elevation view of a tampon product.

### DETAILED DESCRIPTION

The following detailed description refers to the accompanying drawings, which illustrate specific examples described by the disclosure. Other examples having different structures and operations do not depart from the scope of the present disclosure. Like reference numerals may refer to the same feature, element, or component in the different drawings.

Illustrative, non-exhaustive examples, which may be, but are not necessarily, claimed, of the subject matter according the present disclosure are provided below. Reference herein to “example” means that one or more feature, structure, element, component, characteristic and/or operational step described in connection with the example is included in at least one embodiment and/or implementation of the subject matter according to the present disclosure. Thus, the phrase “an example” and similar language throughout the present disclosure may, but do not necessarily, refer to the same example. Further, the subject matter characterizing any one example may, but does not necessarily, include the subject matter characterizing any other example.

The present disclosure relates to a product dispensing system. The system is configured to retain a quantity of one or more products and then dispense the products as needed. It is contemplated that the system may be best suited for dispensing products that are individually packaged goods of uniform size and shape. For example, personal care products and PPE.

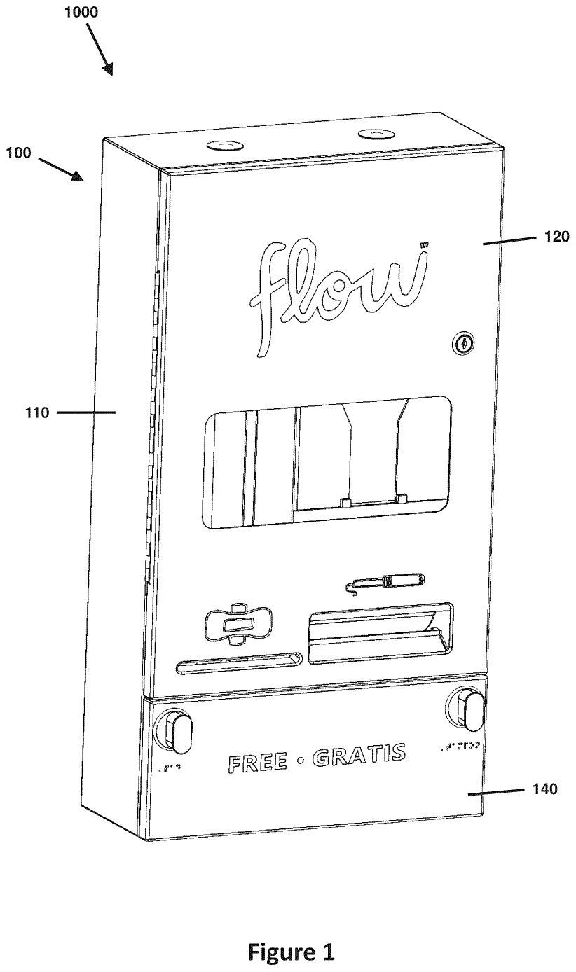

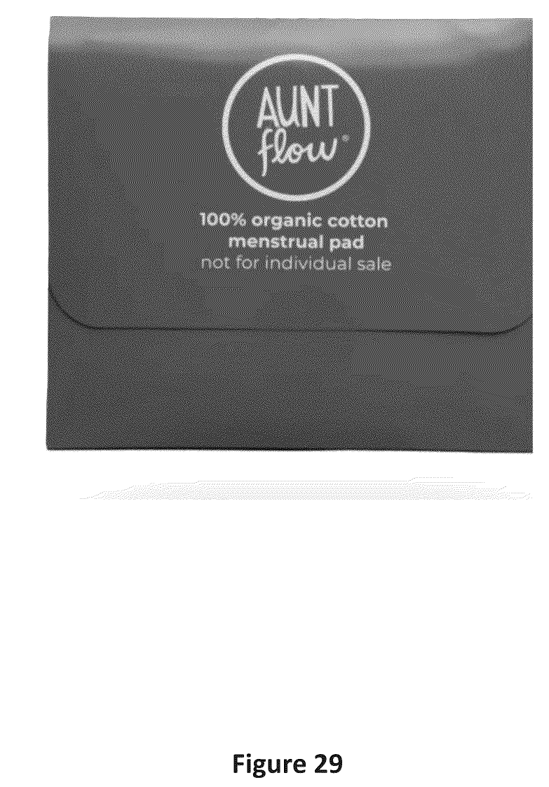

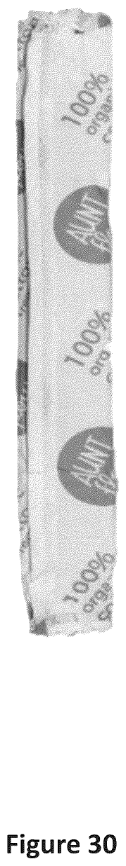

Referring to FIG. 1, depicted is an exemplary embodiment of the system (referred to herein as “system 1000”) that is configured to dispense menstrual pad products and tampon products. As used herein, the term “menstrual pad product” refers to an individual package of a menstrual pad (e.g., FIG. 29); and the term “tampon product” refers to an individual package of a tampon (e.g., FIG. 30). Those skilled in the art will appreciate that menstrual pad products tend to be relatively flat and either square or rectangular in shape. On the other hand, individual packages of tampons tend to be relatively narrow and cylindrical in shape.

Despite the system of FIG. 1 being configured specifically for menstrual pad products and tampon products, in practice, it is contemplated that the system may also be suitable for dispensing other types of products of a similar size and shape.

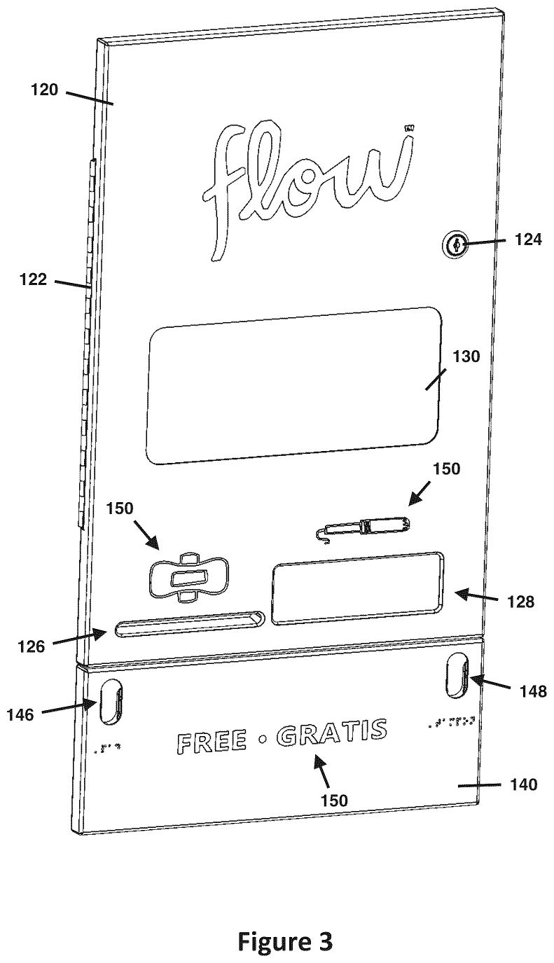

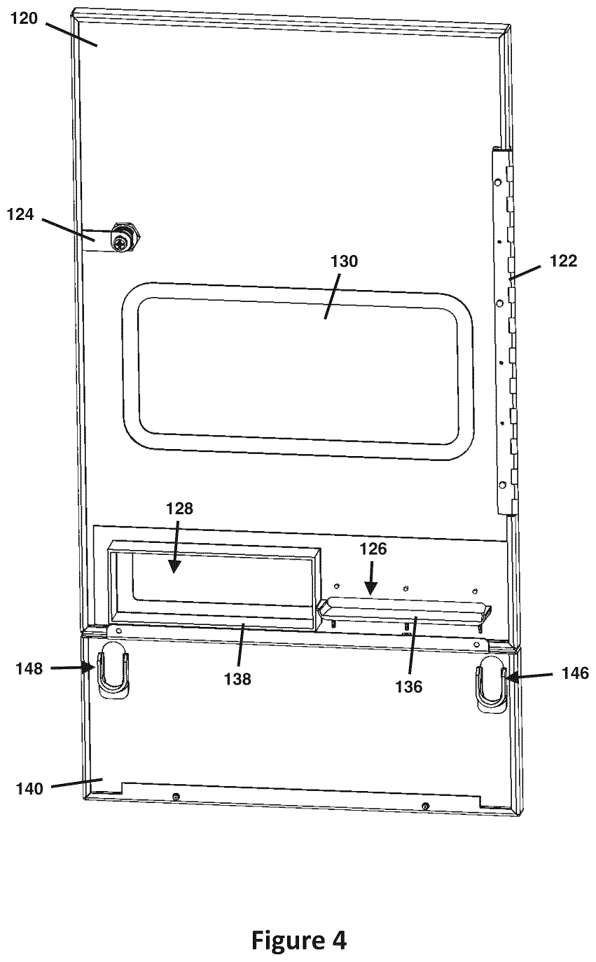

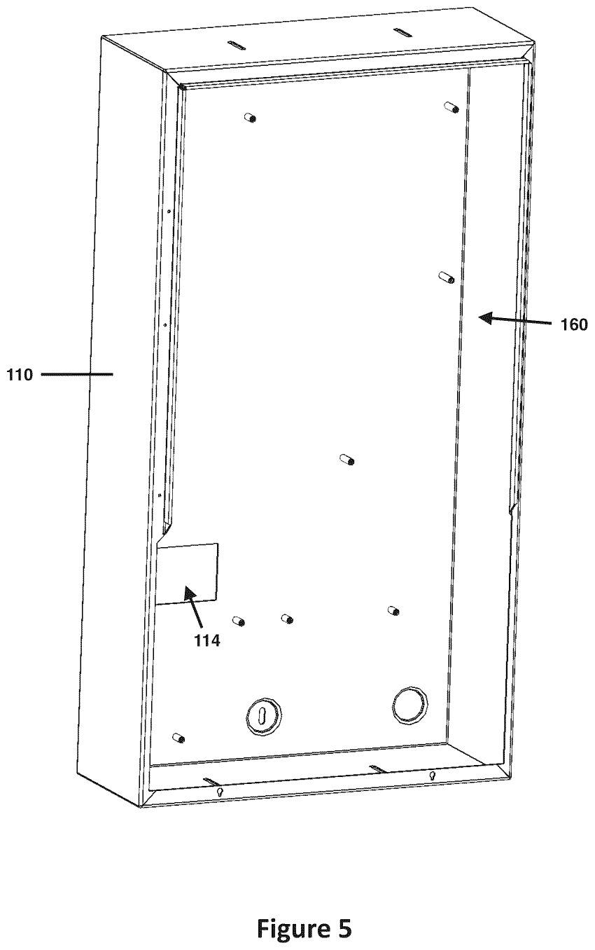

Referring to FIGS. 3-5, system 1000 includes an outer housing 100 that includes rear case 110, removable front cover 120, and paneling 140. Rear case 110 is a vertically elongated five-sided enclosure that defines an open front-side and an interior volume 160 (FIG. 5). Front cover 120 and paneling 140 are attached to the rear case 110 along the open front-side to close off the interior volume 160 (thereby securing the products contained within). In this embodiment, front cover 120 is pivotably attached to rear case 110 via piano-style hinge 122 and secured close via cam lock 124 (best shown in FIG. 4). By unlocking cam lock 124 and pivoting open the front cover 122, access can be granted into the interior volume 160 (e.g., for restocking or other purposes). Those skilled in the art will appreciate that there may be several different suitable means for attaching and/or securing front cover 120 and paneling 140 to rear case 110 (e.g., mechanical fasteners, adhesives, magnets, etc.). Variations such as these will not result in a departure from the scope of the present disclosure.

As those skilled in the art will appreciate, many of the dispensing systems currently in use are located or stored within recesses defined in some sort of structure (e.g., walls, cabinets, etc.). With this in mind, it is contemplated that the dispensing system of the present disclosure (e.g., system 1000) should be relatively compact in size, so as to fit within the same recesses and serve as a possible replacement (i.e., upgrade) for the existing recessed dispensing systems. An exemplary embodiment of the present system may have an outer housing that is, for example, about 26 inches in height, about 14 inches in width, and about 6 inches in depth. Moreover, it is also contemplated that the present system may further include a peripheral flange extending from the outer housing to cover (i.e., hide) any gaps or spaces that result due to any size differences between the present system and an existing to-be-replaced recessed dispensing system (FIG. 31). This peripheral flange may be formed with the outer housing or attached to it as a separate piece.

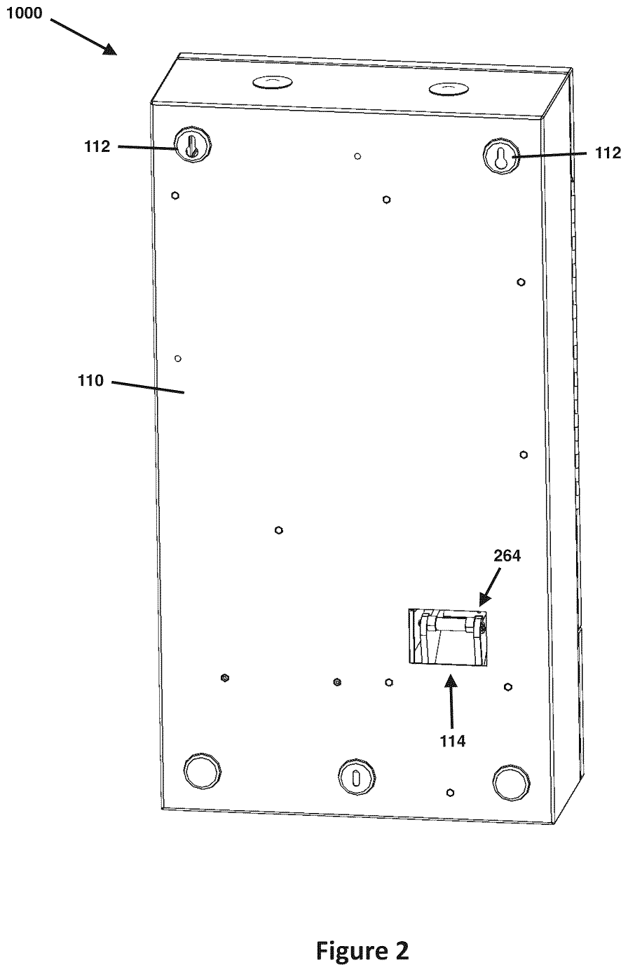

Referring to FIG. 2, the rear case 110 may include a one or more of mounting features that facilitate it being mounted onto a vertical structure, such as a bathroom wall. In the embodiment shown, these mounting features include several keyhole slots 112 incorporated into the rear exterior side of rear case 110. These keyhole slots 112 can be slidably received over, for example, a bolt, a screw, and/or the like.

Referring to FIGS. 3-4, front cover 120 also includes window 130 which may be provided to enable an outside view into the interior volume 160. This may be useful for quickly determining how much product is left in the system 1000 and whether it needs to be refilled. Window 130 may be formed of fully or partially transparent plastic or glass, and may be in a central location on the front cover 120. Moreover, the outer housing 100 may further include markings 150 to decorate the system and/or convey information about the system 1000 such as what types of products it dispenses and whether it costs money. In exemplary embodiments, markings 150 may include brail for vision-impaired persons in compliance with the Americans with Disabilities Act of 1990.

Slots 126 and 128 defined in the front cover 120 allow for products to pass through the outer housing 100. Notably, slot 128 is relatively large and may enable a person to reach into the interior volume 160 to retrieve a dispensed product. In contrast, slot 126 is much narrower and only permits products to be ejected out of the outer housing 100.

Openings 146 and 148 defined in the paneling 140 allow for push-activated buttons 292 and 394 to be incorporated into the outer housing 100 (shown in FIG. 1). These push-activated buttons 292, 294 are provided to enable users to initiate the dispensing of a product (described in greater detail below).

Referring to FIG. 4, either formed along with the front cover 120 or attached as a separate piece, the interior surface of front cover 120 includes trays 136 and 138 located proximate (i.e., at or near) slots 126 and 128. Tray 136 is flush with (i.e., is located at and matches the shape of) the lower edge of opening 126 and can be used to guide product through opening 126 from inside the interior volume 160. Tray 138 is lowered relative to the lower edge of opening 128 and can be used to retain a dispensed product within the interior volume 160 until someone reaches in to retrieve it.

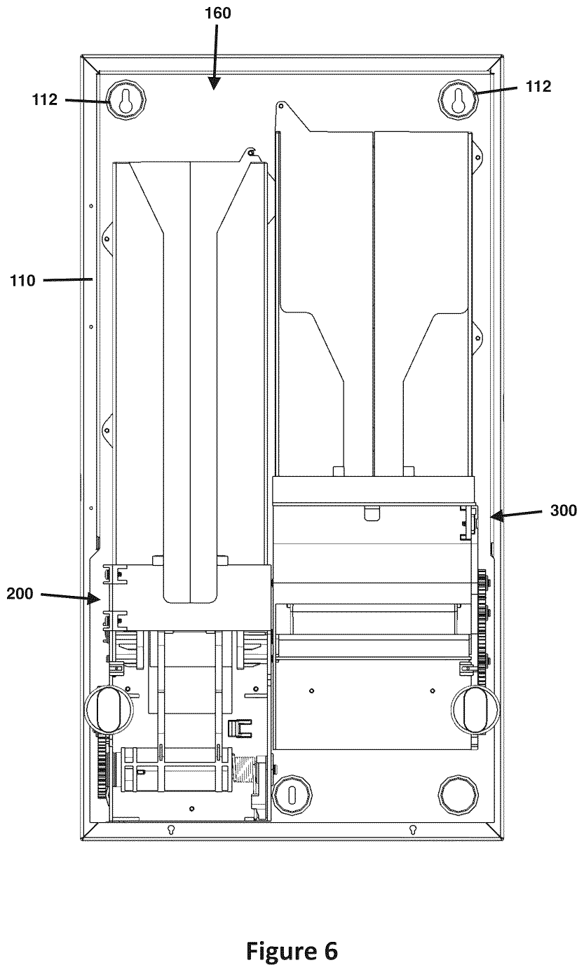

Referring to FIG. 6, system 1000 further includes a first dispensing unit 200 and a second dispensing unit 300 housed within the interior volume 160. Generally speaking, the first and second dispensing units 200, 300 are configured to retain a quantity of product and, when activated, to dispense a product towards a tray (which either retains the product within the interior volume or guides it through an opening in the front cover). In this embodiment, the first dispenser unit 200 is configured to dispense menstrual pad products towards tray 136 and the second dispenser unit 300 is configured to dispense tampon products towards tray 138.

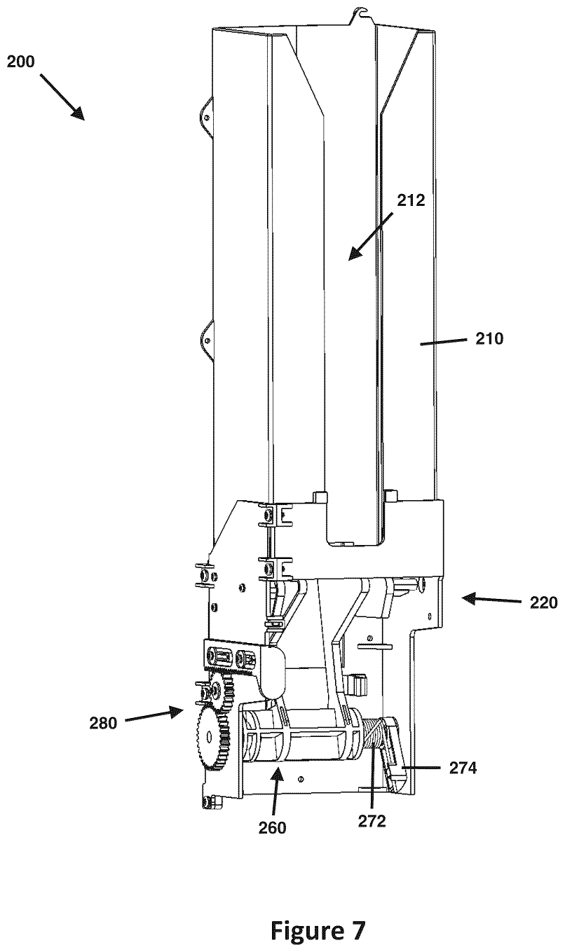

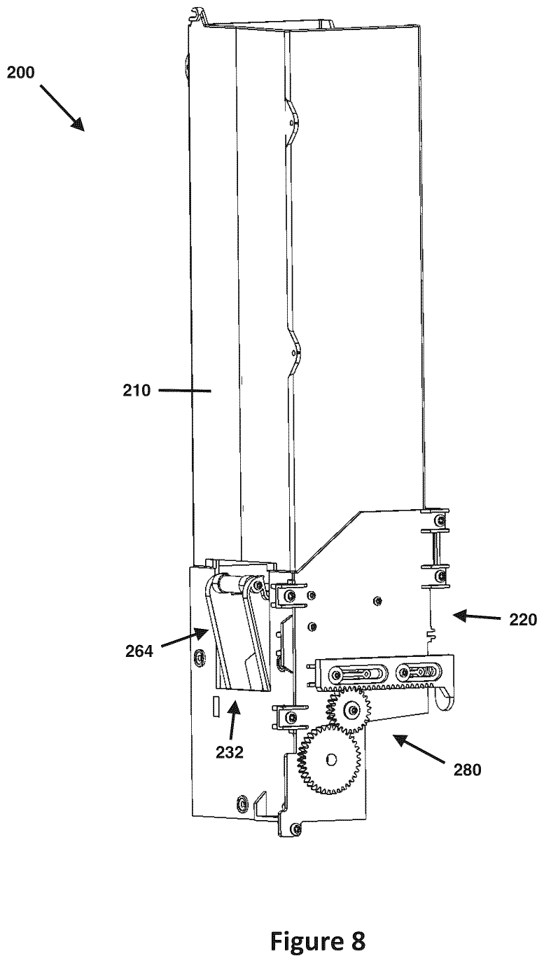

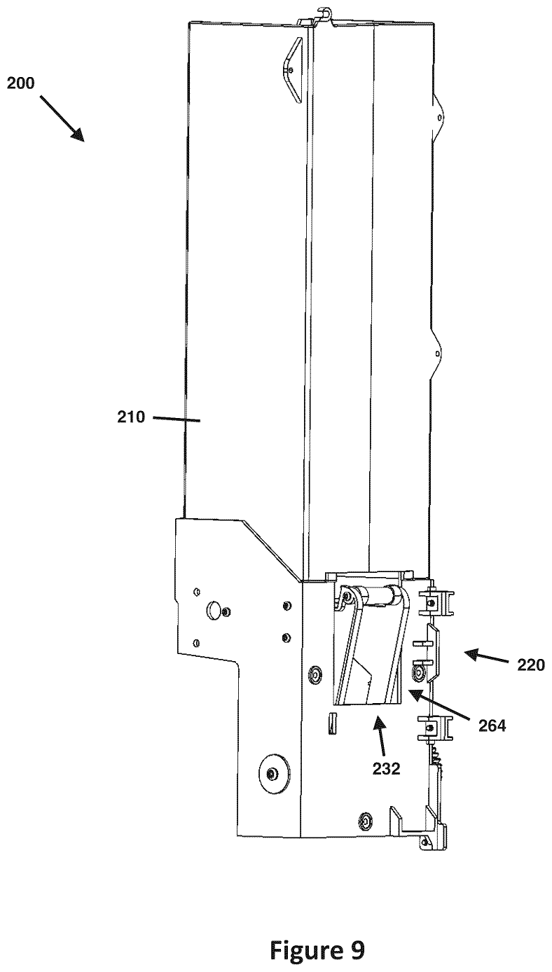

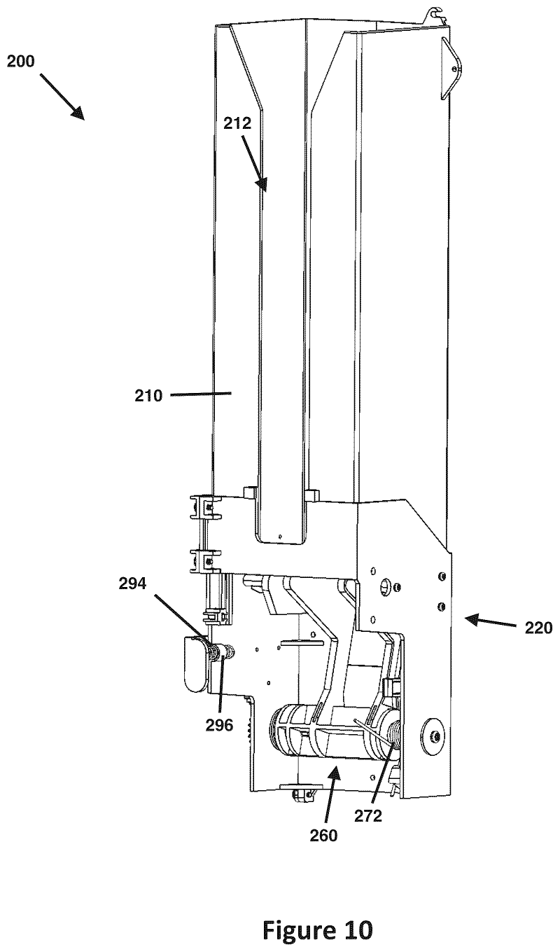

Referring to FIGS. 7-10, the first dispenser unit 200 includes chute 210, chassis 220, rocker component 260 (i.e., moving element), and gear assembly 280.

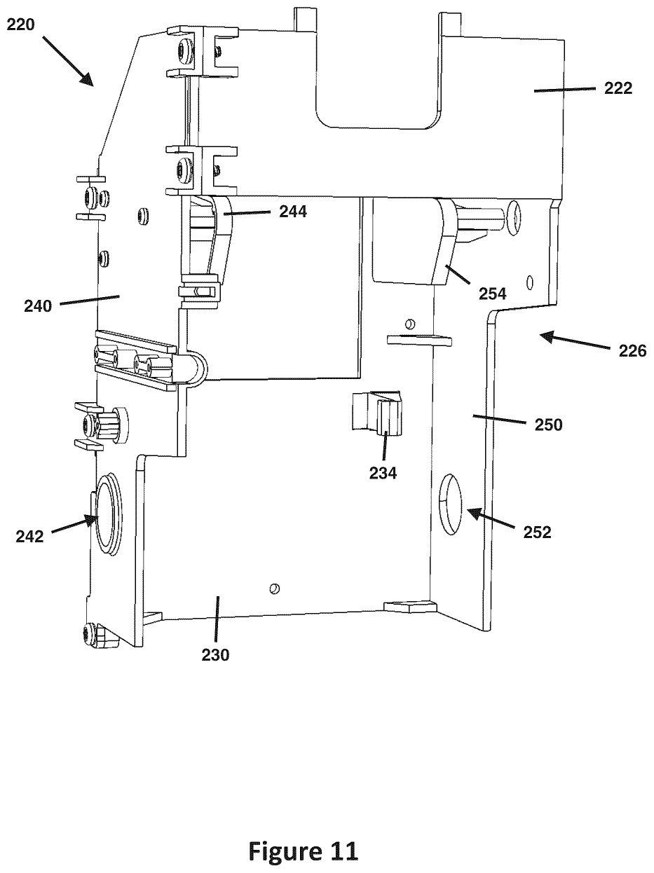

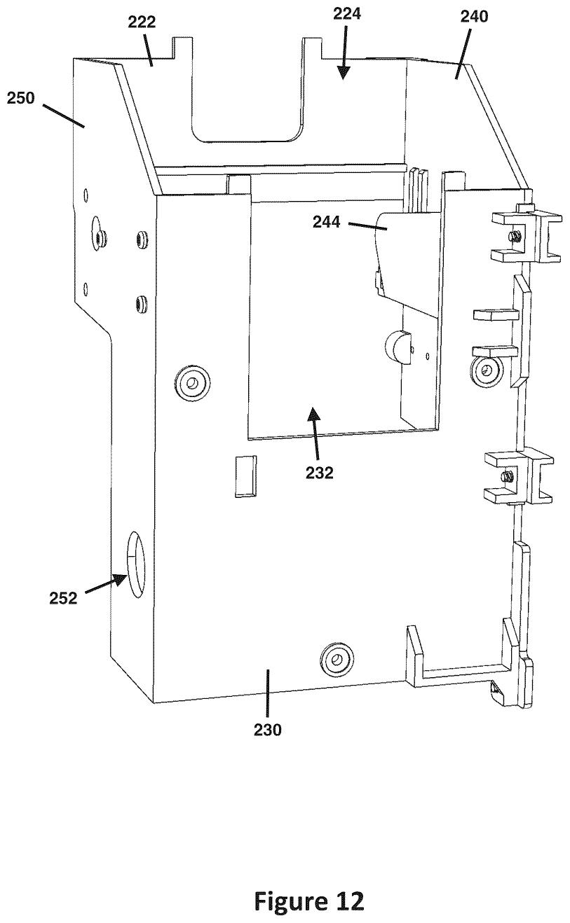

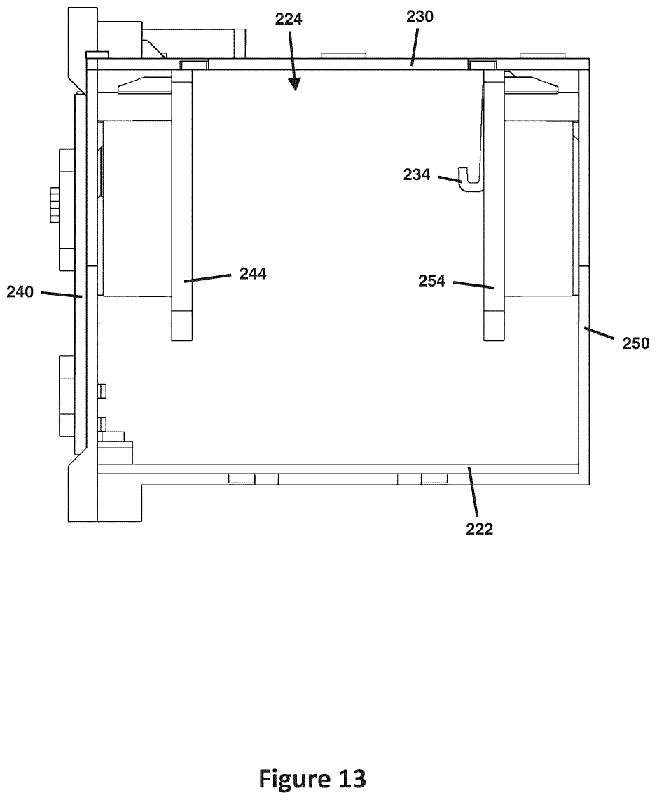

Referring to FIGS. 11-13, chassis 220 features a four-sided design that includes front-side wall 222, rear-side wall 230, left-side wall 240, and right-side wall 250. These walls 222, 230, 240, 250 may be arranged in a square or rectangular shape (best shown in FIG. 13) to generally match the shape of a menstrual pad product. The rear-, right-, and left-side walls 230, 240, 250 of the chassis 220 are each elongated vertically. The front-side wall 222 is comparatively shorter than the other walls 230, 240, 250 and is located high up relative to the rest of the chassis 220. Thus, an open front side 226 is defined below the front-side wall 222.

In the embodiment shown, front-, rear-, and right-side walls 222, 230, 250 are formed as a single integral piece; and left-side wall 240 is attached to the front-and rear-side walls by a plurality of mechanical fasteners. This configuration may allow for left-side wall 240 (and all the dispenser unit components attached to it) to be detached as needed, such as for repair or maintenance purposes.

Near the top of the right-and left-side walls are rails 244 and 254. Rails 244 and 254 may be located proximate the rear wall 230 and may extend towards the interior center of the chassis 220. Notably, these rails 244, 254 define a top-side, rear-to-front downward slope that is angled towards the open front side 266 of the chassis 220. In preparation to be dispensed, a menstrual pad product may be loaded into the chassis 220 by placed it on top of these two rails 244, 254.

Referring to FIG. 7-10, chute 210 can be attached to the top of the chassis 220 and utilized to retain a stack of menstrual pad products above the rails 244 and 254. As the first dispensing unit 200 dispenses product, the stack will continuously be gravity-fed into the chassis 220. Preferably, chute 210 may match the size and shape of the top-side opening 224 (i.e., square or rectangular) and may define a front-side cutout 212 extending along its vertical height to enable people to see the level or quantity of product remaining in the chute.

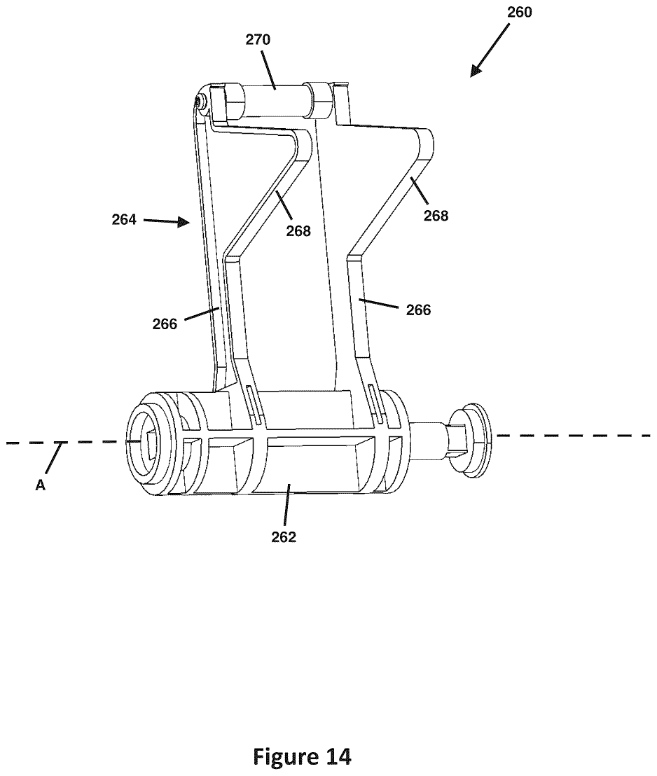

Referring to FIGS. 14-16, rocker component 260 includes linear shaft 262 and rocker arm 264. Linear shaft 262 defines opposing ends which can be fitted into opposing openings 242, 252 defined in the right-and left-side walls 240, 250, below rails 244 and 254 (shown in FIG. 11). Either through a sufficiently loose fit or by incorporation of roller bearings (or similar type of device), linear shaft 262 should be permitted to rotate about its central axis A (shown in FIG. 14) when fitted into the chassis 220.

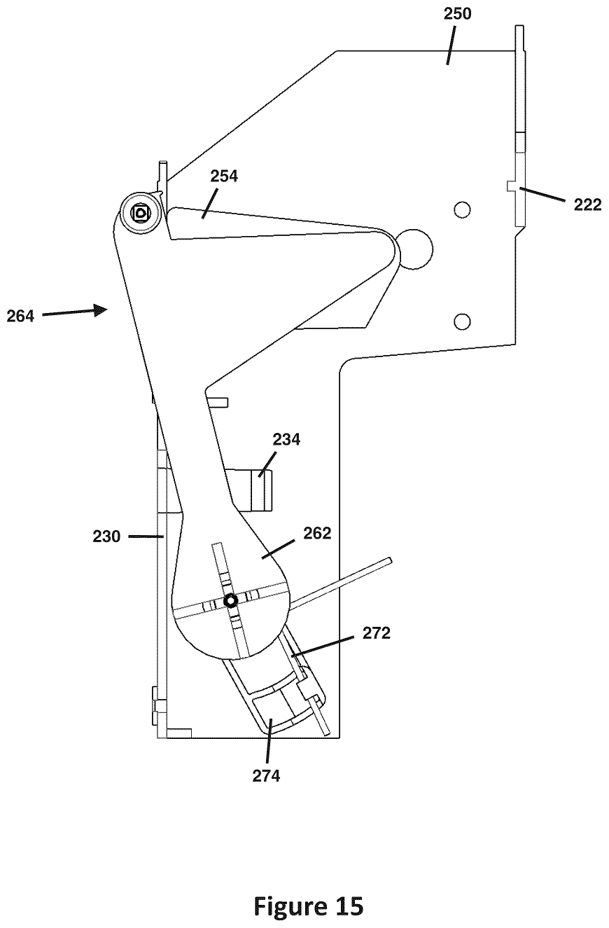

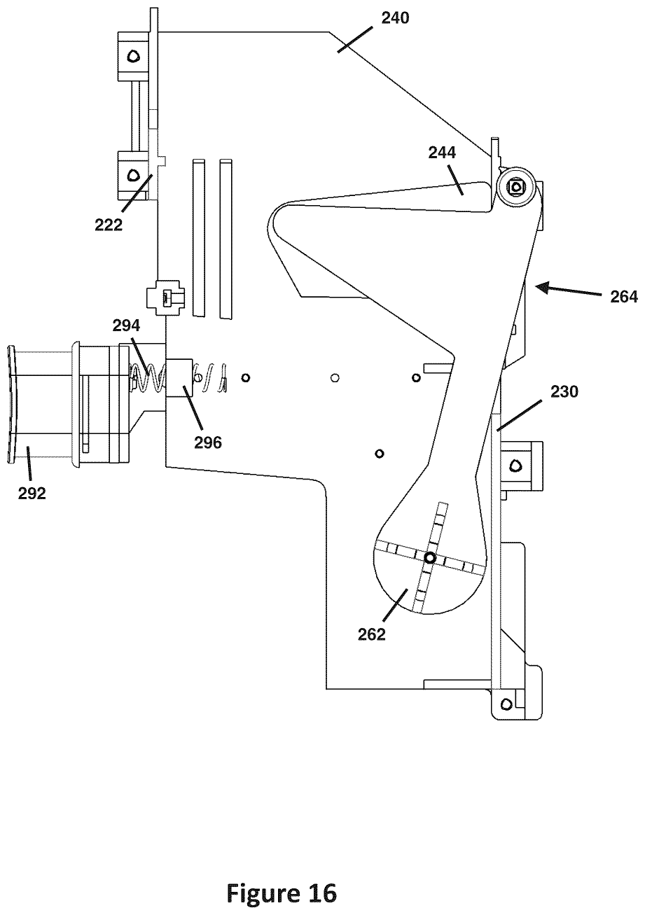

Rocker arm 264 includes two linear segments 266 (i.e., portions) of equal length that extend parallel to one another transverse to the central axis A of the shaft 262. The distal ends of the linear portions 266 are connected by crossmember 270. Near the distal ends of the linear portions 266, but below crossmember 270 (i.e., at a closer radial distance to shaft 262), the rocker arm 264 also includes forward protrusions 268. Forward protrusions 268 extend towards the front-side of the chassis 220 may be utilized to support the center of a menstrual pad product from below once it has been loaded onto rails 244 and 254 (FIGS. 15-16).

Rotation of the linear shaft 262 causes the rocker arm 264 to rock (i.e., pivot) back and forth within the chassis 220 between at least a first position and a second position. In the first position, the distal ends of linear segments 266 will be oriented towards the rear wall 230 behind rails 244 and 254 (FIGS. 15-16). Cutouts 232 and 114 may be defined in the rear wall 230 and the rear case 110, respectively, to enable the linear segments 266 to pivot further backwards as needed. In the second position, the distal ends of linear segments 266 will be oriented forward, towards front-side wall 222. To set a limit on the rocker arm's 264 forward range of motion, hook 234 or a similar type of structure may be provided to catch or stop at least one of the linear segments 266.

The movement of the rocker arm 264 from it first position to its second position enables it to push a menstrual pad product resting on rails 244 and 254 towards the open front side 226 of the chassis 220. Thus, the chassis 220 should be oriented within the interior volume 160 such that its open front side 226 is facing tray 136, and positioned close enough to tray 136 such that a menstrual pad product may be pushed onto it.

In exemplary embodiments, a torsion spring 272 may be received over shaft 262 and fitted into backing 274 (which may be fixedly connected to right-side wall 250). The torsion spring 272 may be configured urge the rocker arm 264 back towards its first position so that it is ready to dispense another menstrual pad product.

In exemplary embodiments, crossmember 270 may include a rolling element configured to roll underneath a menstrual pad product when the linear segments are moved (e.g., a rod inserted through a roller, or a linear bearing, or a similar type of device). The rolling element may be provided to facilitate the dispensing of a single menstrual pad product (i.e., the one resting on rails 244 and 254) without displacing the menstrual pad product stacked above it.

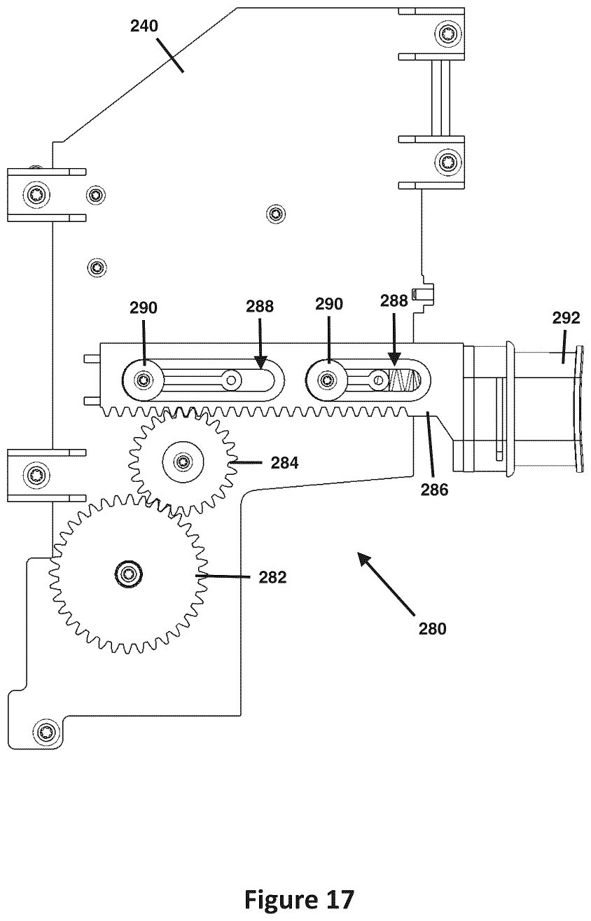

Referring to FIG. 17, the first dispenser unit 200 further includes gear assembly 280 which is configured to move the rocker arm 264 between its first and second positions. Gear assembly 280 is mounted to the exterior surface of left-side wall 240 and includes spur gears 282 and 284, and rack gear 286. Spur gear 282 is fixedly connected to linear shaft 262 and is configured to rotate shaft 262 when turned (i.e., is a drive gear). Rack gear 286 is attached the to the chassis 220 via bolts 290 inserted through slotted holes 288 defined in the rack gear body. The rack gear 286, due to the slotted-hole-and-bolt arrangement, is afforded a linear forwards-and-backwards horizontal range of motion. Spur gear 284 is meshed with rack gear 286 and spur gear 282; and is configured to translate linear motion by the rack gear 286 into the rotational motion of spur gear 282. Thus, when rack gear 286 is pushed backwards, spur gears 282 and 284 may collectively act to pivot rocker arm 264 towards its second position (thereby causing it to dispense a menstrual pad product). The rocker arm 264 returns to its first position when rack gear 286 is moved in the forward direction.

Rack gear 286 defines a forward end that extends past the front-side wall 222 of the chassis 220. Button 292 may be attached to, or formed as a part of, this forward end and may be fitted through opening 146 defined in the paneling 140. This configuration enables a person to activate or actuate the first dispenser unit 200 from outside of the outer housing 100. Preferably, button 292 may be spring backed (e.g., by spring 294 and backing 296, shown in FIG. 16) to urge the rack gear 286 forward and, by extension, the rocker arm 264 towards its first position.

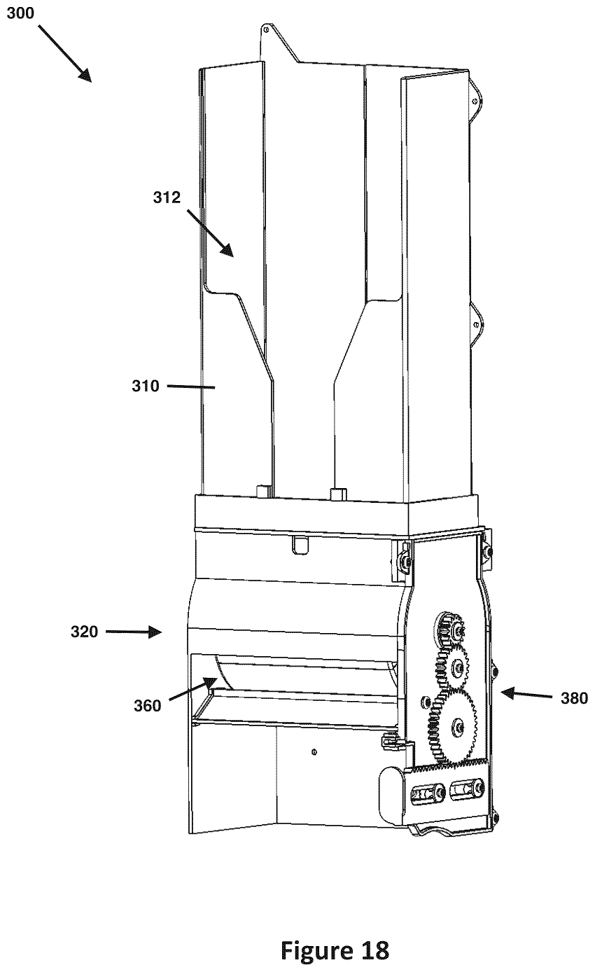

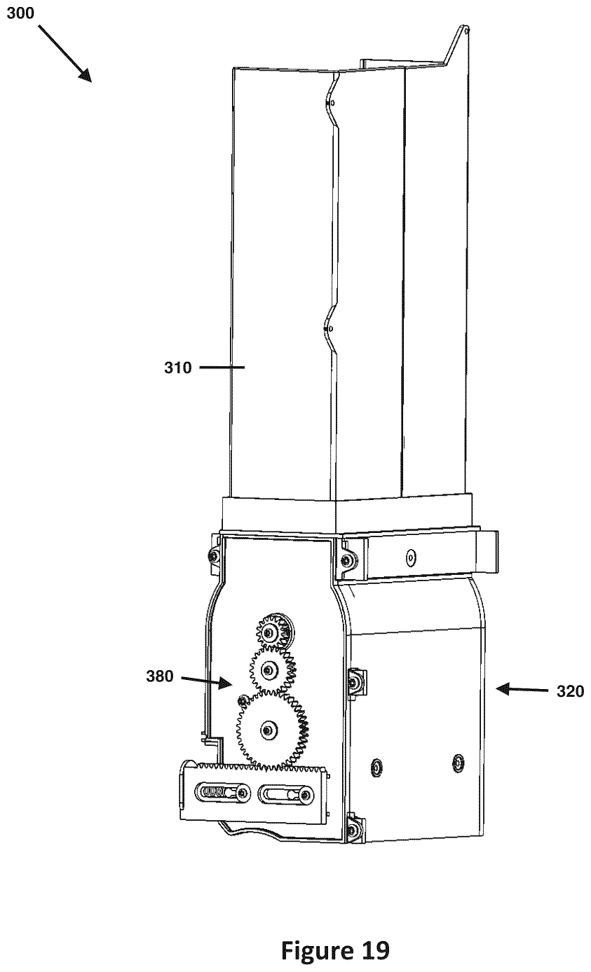

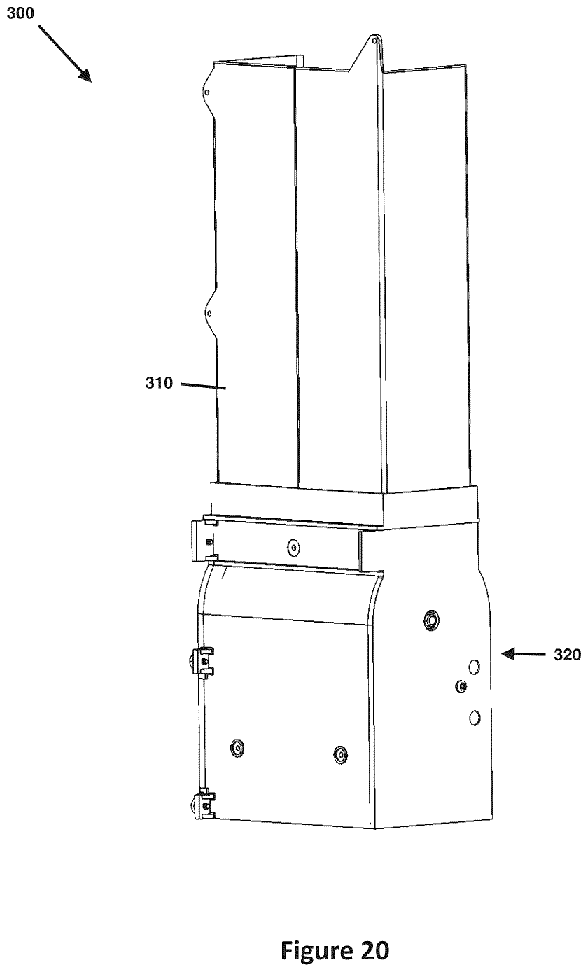

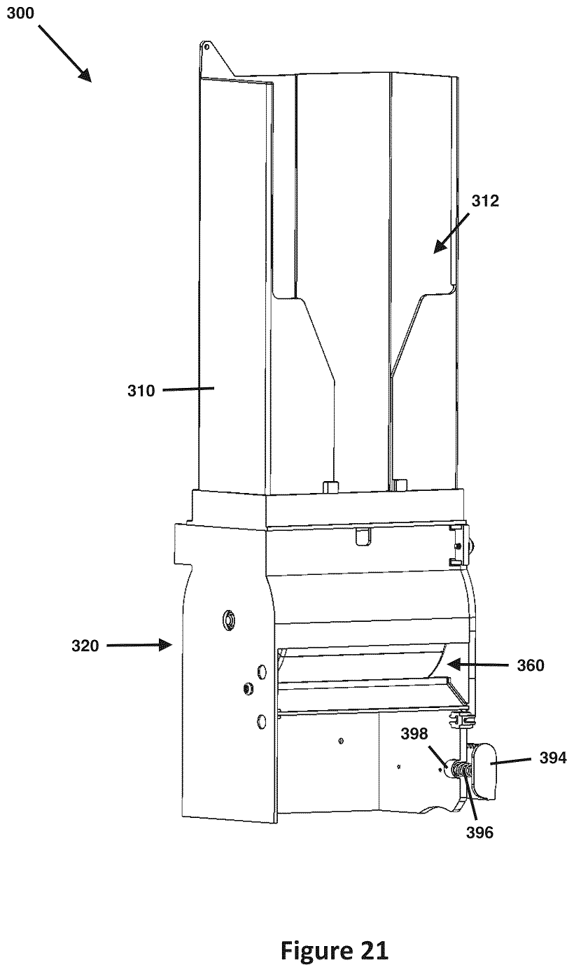

Referring to FIGS. 18-21, the second dispenser unit 300 includes chute 310, chassis 320, roller component 360 (i.e., moving element), and gear assembly 380.

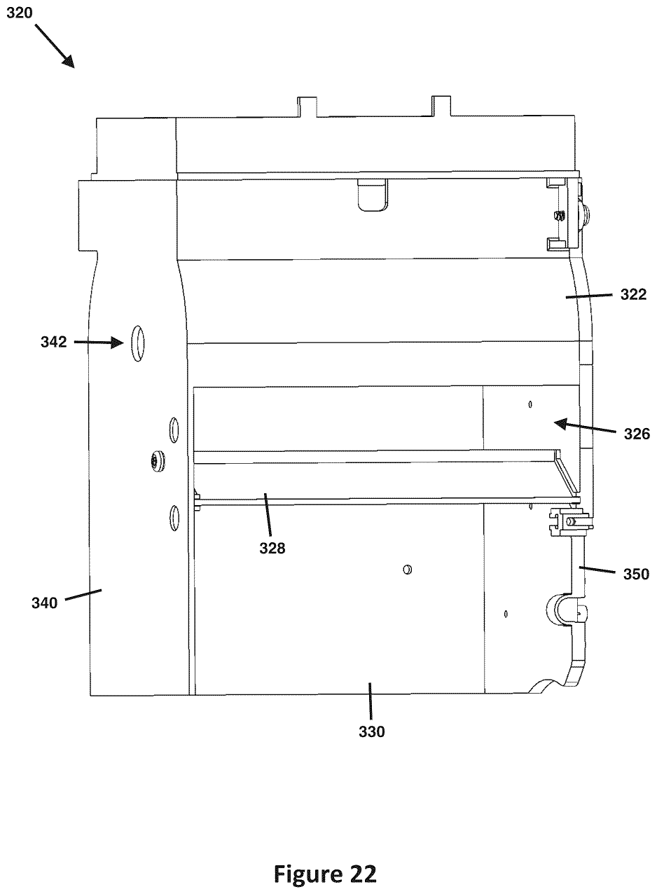

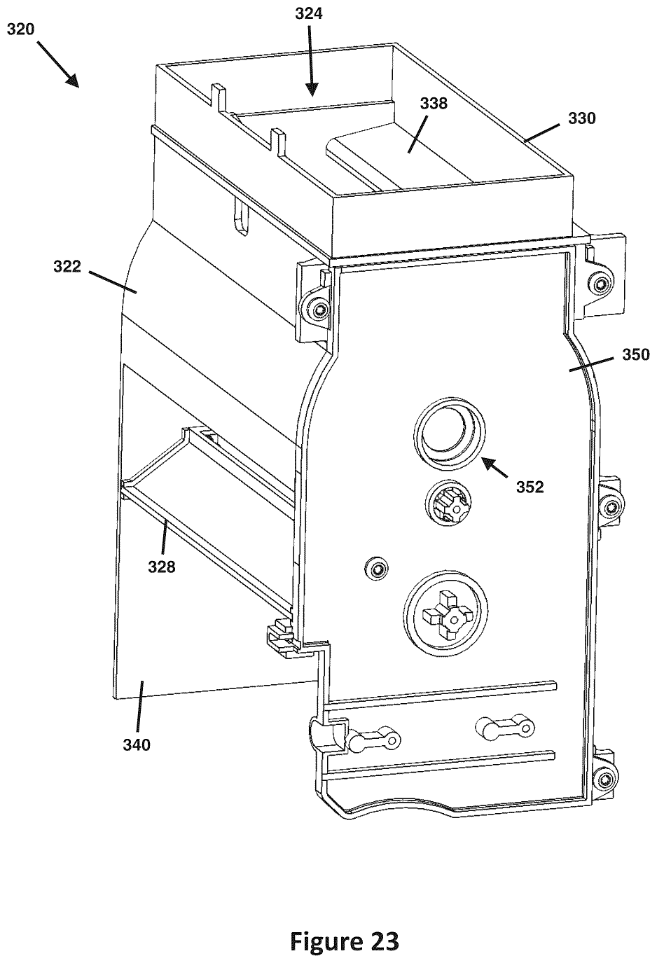

Referring to FIGS. 22-23, the chassis 320 features a four-sided design that includes front-side wall 322, rear-side wall 330, left-side wall 340, and right-side wall 350. The rear-, right-, and left-side walls 330, 340, 350 of the chassis 320 are each elongated vertically. The front-side wall 322 is comparatively shorter than the other walls 330, 340, 350 and is located high up relative to the rest of the chassis 320, resulting in an open front side 326 below the front-side wall 326. Further, the walls of the chassis 320 also define a top-side opening 324 through which tampons can be loaded into the chassis 320.

In the embodiment shown, front-, rear-, and left-side walls 322, 330, 340 are formed as a single integral piece; and right-side wall 350 is attached to the front-and rear-side walls 322, 330 by a plurality of mechanical fasteners. This configuration may allow for right-side wall 350 (and all the dispenser unit components attached to it) to be detached as needed, such as for repair or maintenance purposes.

Referring to FIGS. 18-21, chute 310 can be attached to the top of the chassis 320, fitted over the top-side opening 324, and utilized to retain a quantity of tampon products so that they may be gravity-fed into the chassis 320. Preferably, chute 310 may match the size and shape of the top-side opening 324 and may define a front-side cutout 312 extending along its vertical height to enable people to see the level or quantity of product remaining in the chute 310.

Referring to FIG. 23, the chassis 320 also includes ramps 328 and 338. Ramp 338 is disposed near the top of rear-side wall 330 and extends across its entire width. Ramp 338 defines a rear-to-front downward slope and, in practice, serves to direct tampon products towards the interior front of chassis 320. On the other hand, ramp 328 is located along the open front side 326 of the chassis 320, below front wall 322, and extends between the right-and left-side walls 340, 350. Ramp 328 defines a rear-to-front downward slope and, in practice, serves to direct dispensed tampon products out of the chassis 320 through the open front side 326.

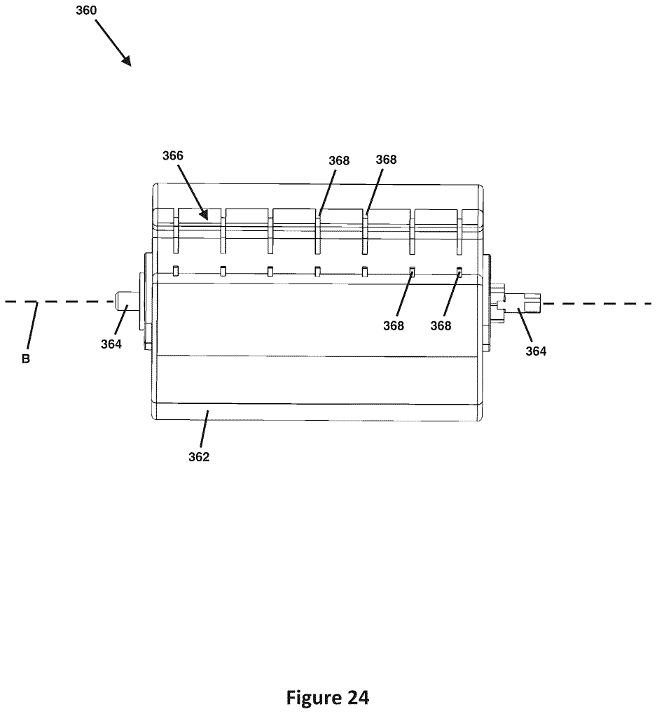

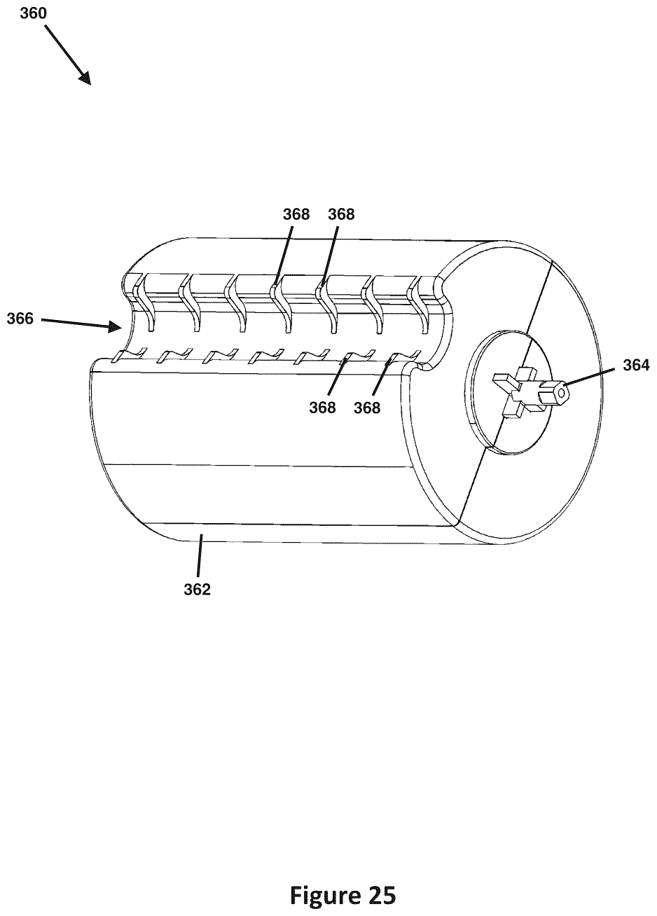

Referring to FIGS. 24-25, roller component 360 includes a cylindrical portion 362 that is elongated along central axis B. Roller component 360 also includes opposing axial portions 364 that extend from the opposing ends of the cylindrical portion 362. These opposing axial portions 364 can be fitted into opposing openings 342 and 352 defined in the right-and left-side walls 340, 350 (shown in FIGS. 22-23). Either through a sufficiently loose fit or by incorporation of roller bearings (or similar type of device), roller component 360 should be permitted to rotate about central axis B when fitted into the chassis 320.

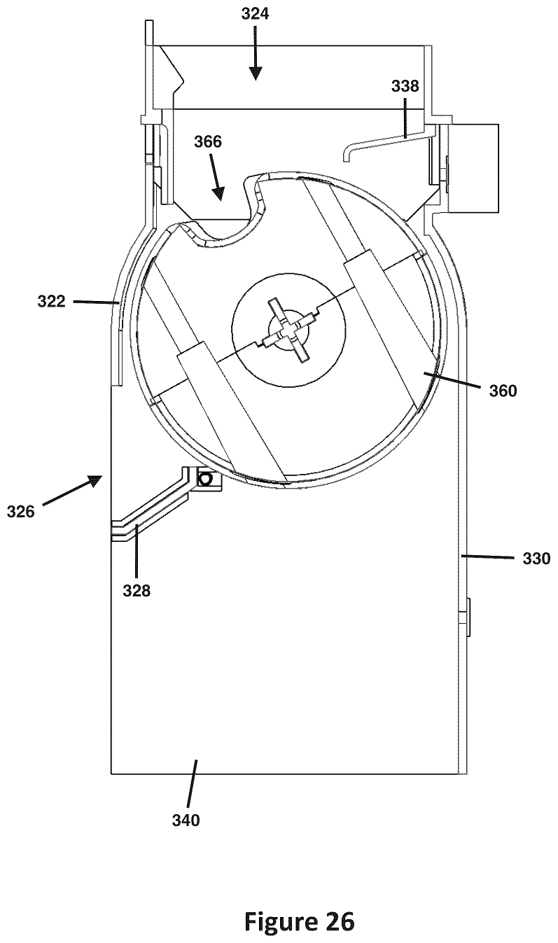

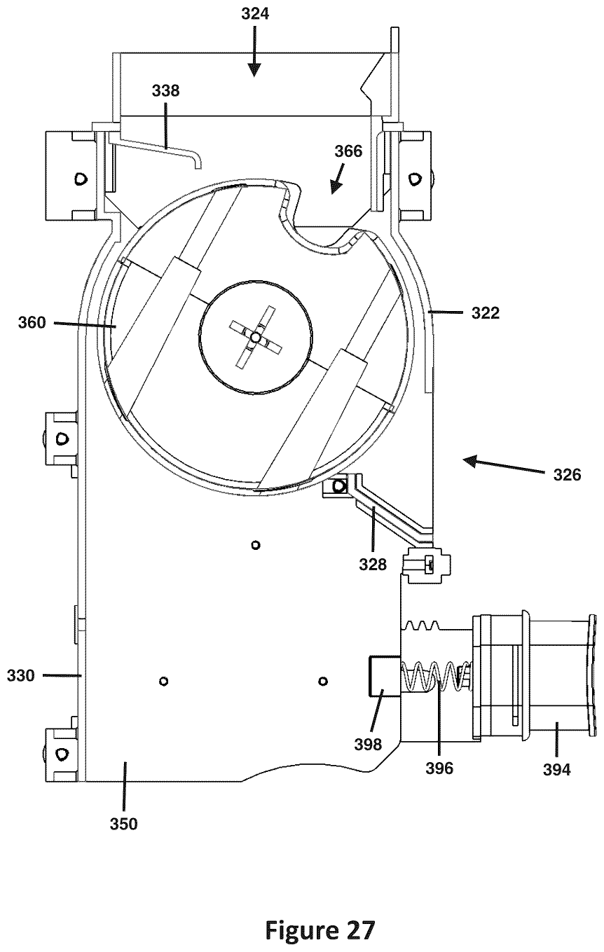

The cylindrical portion 362 defines a linear groove 366 in its exterior surface that extends parallel to central axis B (in this embodiment, from end to end). Referring to FIGS. 26-27, rotation of the roller component 360 causes the groove 366 to move back and forth between at least a first angular position and a second angular position. In the first angular position, the groove 366 will be oriented upwards, facing the top-side opening 324. In the second angular position, the groove 366 will be oriented further downwards, in-between front-side wall 322 and ramp 328.

In practice, roller component 360 may be rotated into its first position to allow a tampon product to roll or fall (i.e., be loaded) into the groove 366. Ramp 338 facilitates the loading of groove 366 by urging or directing tampon products towards it. As needed or desired, a plurality of ribs 368 (or a similar type of structure) may be included within the groove 366 to limit the available space within it, thereby also limiting the number of tampon products capable of fitting within the groove 366 any given time (e.g., to only one or two). From there, the roller component 360 may be rotated into its second position to deliver the tampon product to the open front side 326 of the chassis 320. The tampon product may then fall out of the groove 366 and onto ramp 328 (thereby being dispensed). Thus, it is contemplated that the position of chassis 320 within the interior volume 160 should be close enough to tray 138 such that a dispensed tampon product may roll off of ramp 328 and land on top of it.

As shown in FIGS. 26-27, the upper portion of chassis 320 may be narrower relative to its bottom portion. The narrowing of the upper portion may generally correspond with the curvature of roller component 360. This limits the degree of clearance (i.e., spacing) between the front-and rear-side walls 322, 330 and roller component 360 and prevents tampon products retained above the roller component 360 from accidentally falling below it.

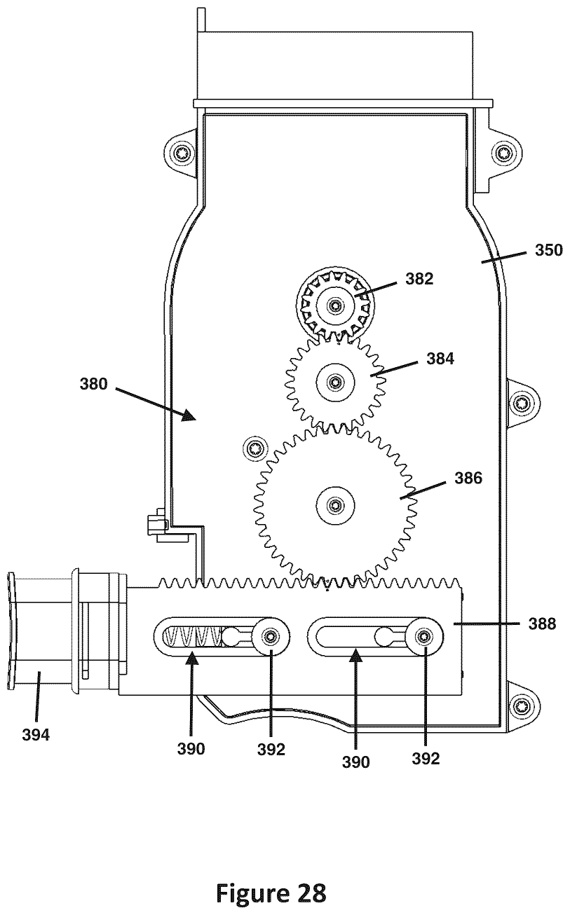

Referring to FIG. 28, the second dispenser unit 300 further includes gear assembly 380 which is configured to move the roller component 360 between its first and second angular positions. Gear assembly 380 is mounted to the exterior surface of right-side wall 350 and includes spur gears 382, 384, and 386, and rack gear 388. Spur gear 382 is fixedly connected to the right-side axial portion 364 of the roller component 360 and is configured to rotate cylindrical portion 360 when turned (i.e., is a drive gear). Rack gear 388 is attached the to the chassis 320 via bolts 392 inserted through slotted holes 390 defined in the rack gear body. The rack gear 388, due to the slotted-hole-and-bolt arrangement, is afforded a linear forwards-and-backwards horizontal range of motion. Spur gears 384 and 386 are meshed with each other and with rack gear 388 and spur gear 382. Spur gears 384 and 386 are configured to translate linear motion by the rack gear 388 into the rotational motion of spur gear 382. Thus, when rack gear 388 is pushed backwards, spur gears 382, 384, and 386 may collectively act to rotate roller component 360 from its first angular position towards its second angular position and cause it to dispense a tampon product. The roller component 360 returns to its first angular position when rack gear 388 is moved in the forward direction.

Rack gear 388 defines a forward end that extends past the front-side wall 322 of the chassis 320. Button 394 may be attached to, or formed as a part of, this forward end and may be fitted through opening 148 defined in the paneling 140. This configuration enables a person to activate or actuate the second dispenser unit 300 from outside of the outer housing 100. Preferably, button 394 may be spring backed (e.g., by spring 396 and backing 398, shown in FIG. 27) to urge the rack gear 388 forward and, by extension, the roller component 360 towards its first angular position.

As those skilled in the art will appreciate, locating the gear assemblies 280, 380 of the first and second dispensing units 200, 300 on opposite side walls of their respective chassis (left-side wall 240 for the first dispensing unit 200, right wall 350 for the second dispensing unit 300) allows for the first and second dispensing units 200, 300 to be positioned relatively close to one another within the other housing 100. Moreover, the gear assemblies 280, 380 themselves—both of which consist of generally planar spur gears and a generally planar rack gear—do not occupy much horizontal width. It is contemplated that these two features enable the first and second dispensing units 200, 300 to fit within a relatively small outer housing 100, and therefore also contribute to the overall compactness of the system 1000.

Those skilled in the art will appreciate that although the embodiment shown in FIGS. 1-28 is considered exemplary, it is not meant to be limiting. Variations can be made to this design without departing from the scope of the present disclosure. Described below are several such variations.

It is contemplated that other embodiments of the system may include either more or less dispensing units than the embodiment shown in FIGS. 1-28, thereby enabling these systems to dispense either more or less types of products. Accordingly, trays and front cover openings can be provided and defined as needed to accommodate varying numbers of dispensing units. Likewise, the sizing and shape of the outer housing may also be varied to adequately house these dispensing units.

While the system described herein includes free-vend devices (those that do not require payment to be received in order to dispense a product), one of ordinary skill in the art will recognize that different embodiments could easily be configured to require payment before dispensing one or more products. Required payment could be in a variety of forms including but not limited to paper money, coins, and card payments. In some embodiments a dispensing unit may require payment prior to dispensing one type of product but be configured as free-vend for another type of product.

While in some embodiments the dispensing unit and one or more assemblies therein are manually operated, in other embodiments the dispensing units may be electronically operated. For example, and without limitation, the dispensing units may include electronic componentry sufficient to allow actuation to be initiated by a motion sensor, or by receiving a signal over a network. Motion sensors on a dispensing unit may allow a product to be dispensed to a user in response to the wave of a hand or other movement near the unit. In some embodiments with two or more dispensing assemblies within a single unit, one or more of the dispensing assemblies may be manually operated with one or more other assemblies are electronically operated.

The system and its various components, either individually or collectively, may be manufactured from a variety of materials, including plastic and metal. In exemplary embodiments, many of the components of the system may be formed from high density polyethylene. In exemplary embodiments, the outer housing (e.g., rear case 110, front cover 120, and paneling 140) may be formed from stainless steel.

Any embodiment of the present invention may include any of the features of the other embodiments of the present invention. The exemplary embodiments herein disclosed are not intended to be exhaustive or to unnecessarily limit the scope of the invention. The exemplary embodiments were chosen and described in order to explain the principles of the present invention so that others skilled in the art may practice the invention. Having shown and described exemplary embodiments of the present invention, those skilled in the art will realize that many variations and modifications may be made to the described invention. Many of those variations and modifications will provide the same result and fall within the spirit of the claimed invention. It is the intention, therefore, to limit the invention only as indicated by the scope of the claims.

What is claimed is:

1. A dispensing system comprising:
an outer housing comprising a front cover and a rear case, wherein the rear case defines an interior volume and the front cover defines a slot extending into the interior volume;
a tray located within the interior volume, proximate to the slot;
a dispensing unit housed within the interior volume, the dispensing unit comprising:
a chassis that defines a top-side opening, an open front-side, and an interior space, wherein the open front-side of the chassis is facing the tray, and wherein a product may be loaded into the chassis by being inserted into the top-side opening;
a moving element mounted to the chassis within the interior space and below the top-side opening, the moving element being moveable between at least a first position and a second position, and wherein movement of the moving element from its first position to its second position causes the moving element to move a loaded product towards the tray through the open front side;
a gear assembly configured to move the moving element between its first and second positions, the gear assembly comprising:
a drive spur gear fixedly connected to the moving element;
a rack gear slidably attached to the chassis that is movable along a linear path;
at least one additional spur gear meshed with the drive spur gear and the rack gear, the at least one additional spur gear being configured to translate linear motion by the rack gear into rotational motion of the drive spur gear.

2. The dispensing system of claim 1, wherein:
the moving element is a rocker component that comprises a linear shaft rotatably mounted to the chassis below the top-side opening and a linear section extending transverse to the linear shaft;
in the first position, the linear section is oriented upwards towards the rear of the chassis;
in the second position, the linear section is oriented upwards towards the open front side;
movement of the rocker component from its first position to its second position causes the linear section to push a loaded product towards the tray through the open front side; and
the drive spur gear is fixedly connected to the linear shaft and is configured to rotate the linear shaft when it is rotated.

3. The dispensing system of claim 2, wherein the linear section of the rocker component defines a distal end, and wherein the rocker component further comprises a rolling element connected to the distal end.

4. The dispensing system of claim 2, wherein the rocker component further includes a forward protrusion extending from the linear section towards the front side of the chassis, the forward protrusion being configured to support a loaded product from beneath.

5. The dispensing system of claim 2, wherein the rocker component further comprises a torsion spring fitted over the linear shaft that is configured to urge the rocker component towards its first position.

6. The dispensing system of claim 2, wherein the chassis further comprises a rail extending into the interior space near the top-side opening, the rail being configured to support a loaded product from beneath.

7. The dispensing system of claim 1, wherein:
the moving element is a roller component that comprises:
a cylindrical portion that defines opposing ends and a curved exterior surface;
opposing axial portions extending from the ends of the cylindrical portion that are rotatably mounted to the chassis;
a groove defined in the exterior surface of the cylindrical portion;
in the first position, the groove is oriented upwards towards the top-side opening of the chassis and is thereby able to receive a loaded product;
in the second position, the groove is oriented at least slightly downwards towards the open front side of the chassis;
movement of the roller component from its first position to its second position causes the cylindrical portion to deliver a product from the top-side opening to the open front side, causing the product to fall out of the groove towards the tray;
the drive spur gear is fixedly connected to an axial portion of the roller component and is configured to rotate the axial portion when it is rotated.

8. The dispensing system of claim 7, wherein the roller component further comprises a plurality of ribs within the groove that limit the number of products capable of fitting within the groove.

9. The dispensing system of claim 7, wherein the chassis further comprises a ramp located along its open front side that defines a rear-to-front downward slope and is configured to direct a product falling out of the groove towards the tray.

10. The dispensing system of claim 1, wherein the slot defined in the front cover defines a lower edge, and wherein the tray is flush with the lower edge of the slot.

11. The dispensing system of claim 1, wherein the wherein the slot defined in the front cover defines a lower edge, and wherein the tray is located below the lower edge.

12. The dispensing system of claim 1, wherein the outer housing defines an opening into the interior volume, and wherein the dispensing system further comprises a button that is attached to the rack gear and fitted into the opening defined by the outer housing.

13. The dispensing system of claim 9, wherein the button is spring-backed and configured to move the rack gear towards one direction along its linear path.

14. The dispensing system of claim 1, wherein the dispensing unit further comprises an elongated chute located above the chassis that is configured to hold a quantity of product and gravity-feed the product into the top-side opening.

15. The dispensing system of claim 10, wherein the chute defines a front-side cutout extending along its length.
